(19) United States
(12) Patent Application Publication
(10) Pub. No.: US 2011/0282456 A1
(43) Pub. Date: Nov. 17, 2011

(54) IMPLANTABLE DEVICE FOR SEALING A SPINAL ANNULAR FISSURE TEAR AND METHOD FOR DEPLOYING THE SAME

(75) Inventors: Roey Shafrir, Modiin (IL); Hamid Sharim, Kokhav Yair (IL)

(73) Assignee: NEWVERT LTD., Netanya (IL)

(21) Appl. No.: 13/146,403

(22) PCT Filed: Feb. 5, 2010
(86) PCT No.: PCT/IB10/50536

§ 371 Date: Jul. 27, 2011

Related U.S. Application Data
(60) Provisional application No. 61/150,014, filed on Feb. 5, 2009.
(60) Provisional application No. 61/186,323, filed on Jun. 11, 2009.

(51) Int. Cl. A61F 2/44

(52) U.S. Cl. 623/17.16

(57) ABSTRACT

The present application relates to an implantable device (104) and a method for delivering the implant for the repair and closure of annular defects and in particular, to such a device and method in which spinal annular defects are repaired with an elastic-plastic implant adept to securely seal an annular defect while providing a sufficiently compliant implant that adapts to bodily movements.

### FIELD OF THE INVENTION

The present invention relates to an implantable device and a method for delivering the implant for the repair and closure of annular defects and in particular, to such a device and method in which spinal annular defects are repaired with an elastic-plastic implant adept to securely seal an annular defect while providing a sufficiently compliant implant that adapts to bodily movements.

### BACKGROUND OF THE INVENTION

Back pain is one of the most common and often debilitating conditions affecting millions of people. The severity of back pain may and is often debilitating greatly limiting people in their activity, therefore indirectly affecting a person suffering from back pain in the level of quality of life.

Some forms of back pain result from anatomical pathologies associated with the spinal column. Specifically, pain is often associated with the interverterbral disc. For example, in a slipped disc the disc is pressed against a nerve root causing irritation and pain. The cause of a slipped disc, or disc herniation leading to back pain, sciatica or the like is degredation of the disc. The outer shell of the disc, namely the annulus fibrosus, gradually or suddenly deteriorates forming a fissure, or tear, eventually leading to larger tears and opening in the disc annulus fibrosis.

Such defects in the annulus allow the gelatinous material of the nucleus pulposus to flow out of the nucleus and into the outer aspects of the annulus. The flow of the nucleus pulposus to the outer aspects of the annulus is termed disc bulge or herniation.

Herniation of the nucleus material bulges through the tear in annulus occurs in the posterior portions of the disc, the nerve roots may be directly and physically impinged by the bulge. In more extreme or progressive instances of annular tears, the nuclear material may escape, further causing chemical irritation of the nerve roots. Dependent upon the cause and nature of the disc protrusion, the condition may be referred to as a disc bulge, a herniated disc, a prolapsed disc, a ruptured disc, or, if the protrusion separates from the disc, a sequestered disc.

Although various inter-vertebral surgical procedures are routinely performed to treat back pain caused from herniation of the disc. A standard surgical procedure for excision of the intervertebral disc, the soft, central portion of the disc, nucleus pulposus, bulges out of the outer, fibrous envelope, the annulus fibrosus and is removed, in a procedure called a discectomy. Such a procedure intentionally leaves the remaining nucleus pulposus within the inter-vertebral space. However as a result of such procedure the tear in the annulus fibrosus is commonly not sealed or repaired. The long term effect of such procedures are therefore in question as additional the reoccurrence of disc herniation is not uncommon as the tear leading to the herniation is not treated.

Different solutions in the form or implantable devices, sutures, have been proposed to close the tear as a result of disc degeneration following and/or instead of surgical procedures such as a discectomy. However none have been successful in securely and safely sealing the tear in the disc.

### SUMMARY OF THE INVENTION

The present invention overcomes these deficiencies of the background art by providing an implantable device and a minimally invasive method for delivering the implant to repair and seal a tear in the annulus fibrosus, therein alleviating back pain. The prior art does not provide a long term solution for a displaced disc which results in a recurrence rate of about 10-15% of the patients that suffer from recurring rupturing of the same disc, where each recurring rupture carries the risk of aggravating the fissure and/or increased risk of additional complications. Furthermore the background art does not teach an implant for sealing an annular fibrosus tear by conforming to its anatomy, shape and geometry.

Within the context of this application the term inter-vertebral disc may be interchangeably referred to as a disc.

An optional non limiting embodiment of the present invention optionally and preferably provides for an implant for sealing a tear formed in the intervertebral disc. Optionally the implant according to the present invention comprises a body having a proximal end and distal end wherein the at least one of the proximal and/or distal end comprise an anchor for anchoring the implant to the disc.

An optional non limiting embodiment of the present invention optionally and preferably provides a barrier, for example in the form of an implant, membrane or a combination thereof, for blocking the remaining internal material of the disc from being expelled outside the confines of the annulus fibrosus.

Optionally the implant according to an optional non limiting embodiment of the present invention may be fabricated as a unitary component or from a plurality of components.

Optionally the implant according to an optional non limiting embodiment of the present invention may be adjusted according to at least one or more parameters associated with the implantation site for example including but not limited to size of hernia, size of annular tear, location of hernia, tear size, width-length to any parts of the disc in any angle or the like.

Optionally the implant according to an optional non limiting embodiment of the present invention may be anchored and/or otherwise securely associated with any anatomical structure within the intervertebral disc space for example including but not limited to the vertebral endplate, annulus fibrosus, nucleus pulposus, spinal longitudinal ligaments, any combination thereof, or the like anatomic structure.

Optionally the implant according to an optional non limiting embodiment of the present invention may be anchored in the disc space.

Optionally the implant according to an optional non limiting embodiment of the present invention may be anchored between two vertebrae.

Optionally the implant according to an optional non limiting embodiment of the present invention may comprise at least one or more proximal anchor, optionally disposed within the posterior portion of the disc space.

Optionally the implant according to an optional non limiting embodiment of the present invention may comprise at least one or more distal anchor, optionally disposed in the anterior portion of the disc space.

Optionally the implant according to an optional non limiting embodiment of the present invention may comprise at least one or more distal anchor and at least one or more proximal anchor.

Optionally the implant according to an optional non limiting embodiment of the present invention may comprise at least one or more anchors disposed along the implant body between the distal end and proximal end.

Optionally the implant according to an optional non limiting embodiment of the present invention may comprise at least one or more distal anchor, at least one or more proximal anchor, and at least one or more anchors disposed along the implant body between the distal end and proximal end.

Optionally the implant according to optional non limiting embodiments of the present invention may comprise anchors provided in a plurality of optional forms for example including but not limited to spring, rods, legs, extensions or the like for anchoring the implant to an anatomical location. Optionally the anchors may for example be provided in the form of malleable, flexible, pliable, shape memory components. Optionally the anchors may for example be provided in the form of non flexible, rigid material.

Optionally the implant according to optional non limiting embodiments of the present invention may comprise controllable anchors that may be controllably disposed within an anatomy and/or repositioned within an anatomy.

Optionally the implant according to optional non limiting embodiments of the present invention may be provided in forms that optimize the surface area of the anatomy wherein it is implanted. Optionally the implant may be configured to increase a surface area of the implant within the implanted anatomy, most preferably the annulus.

Optionally the implant according to an optional non limiting embodiment of the present invention may be provided in a plurality of optional forms for example including but not limited to wire mesh, wire braid, wire, group of wires or rods with a flexible core, stent-like tubular structure, bifurcation wire, spring, rods, legs, extensions or the like.

Optionally the implant according to optional non limiting embodiments of the present invention may comprise a body having a controllable shape contour. Most preferably an implant body may be adjusted to correspond with the anatomy and geometry of the annular tear and/or opening wherein it is implanted. Optionally the implant body may assume the form of a longitudinal rod like tube, optionally the tube may for example include but is not limited to a wire mesh, mesh-like structure, braid, polymer mesh or the like flexible and/or pliable material.

Optionally an implant according to optional non-limiting embodiments of the present invention may be provided in an open structure or a closed structure or an intermediate thereof.

Optionally the implant according to optional non limiting embodiments of the present invention may expand to fill the space between two vertebrae.

Optionally the implant according to optional non limiting embodiments of the present invention may be provided in optional forms, for example including but not limited to, flexible due to its irregular profile.

An optional non limiting embodiment of the present invention optionally and preferably provides for an implant having material of varying rigidity for example including but not limited to a substantially flexible implant, partially flexible implant, partially rigid, rigid implant, or the like.

An optional non limiting embodiment of the present invention optionally and preferably provides for an implant comprising a body from about 2 mm to about 40 mm diameter.

An optional non limiting embodiment of the present invention optionally and preferably provides for an implant comprising substantially self expanding materials.

An optional non limiting embodiment of the present invention provides for an implant delivery that is facilitated with at least one or more auxiliary device for example including but not limited to a semi-compliant balloon, medical balloon, balloon propelling screw, endoscope, trocar, catheter, guiding catheter or the like.

Optionally and preferably, during deployment and implant delivery the implant according to optional embodiments of the present invention may be adapted to provide a practitioner with a high degree of freedom for maneuvering the implant therein optionally and preferably allowing a practitioner to reversibly reposition the implant within the implantation site, during delivery process most preferably with damaging tissue. Optionally any portion of the implant may be adjusted, maneuvered and repositioned during implantation so as to give a practitioner full control of the implantation process prior to committing to the implantation. Most preferably the implant and delivery method according to the present invention are both controllable and reversible.

An optional non limiting embodiment of the present invention optionally further comprises at least one or more membrane. Optionally the membrane may be semi-permeable, selectively permeable, permeable, a combination thereof or the like. Optionally at least one membrane may be integrated with an implant according to optional embodiments of the present invention, therein the membrane and implant are optionally implanted simultaneously. Optionally a membrane may be associated with an implant immediately prior to implantation, during implantation or following implantation.

Optionally and preferably at least one or more membrane may be integrated, coupled or otherwise associated with an implant according to optional embodiments of the present invention in at least one or more of its constituents for example including but not limited to the distal portion, proximal portion, medial portions, anchors, body, any combination thereof or the like. Optionally at least one or more membrane may be provided along any surface of the implant for example including but not limited to external surface, internal, intermediate surface, any combination thereof or the like. Optionally an implant according to optional embodiments of the present invention may be associated with at least one or more membranes. Optionally a plurality of membranes may be associated with an implant. Optionally at least one or more membranes may be disposed within an optional implant according to the present invention, optionally the implant may be disposed with variable angles within an implant lumen.

Optionally the membrane may be provided in optional forms for example including but not limited to mesh, foils, polymer sheet, metal sheets, or the like biocompatible material, biological tissue any combination thereof or the like.

Optionally the implant according to optional embodiment of the present invention may be hermetically sealed optionally and preferably in limiting movement of materials through implant body or lumen. Optionally and preferably implant sealing may be provided for example by utilizing at least one or more membrane.

An optional non limiting embodiment of the present invention optionally provides for an implant that may be inserted into the desired position within the disc space, the device may be further anchored into the adjacent vertebral bodies, utilizing screws, nails, hooks, or other such anchoring devices known in the art.

An optional non limiting embodiment of the present invention wherein all materials are medical grade may be made from metal and/or metal alloys for example including but not limited to NiTinol, memory shape alloys, ePTFE and/or polyurethane, silicone, collagen, or the like. Optionally, the implant may be made from biological tissue such as nucleus pulposus, annulus fibrosus, tendon, fascia, bone, or the like. Optionally such biological tissue may be provided from optional sources for example including but not limited to autograft, allograft, xenograft or engineered in laboratory conditions.

Optionally, the implant according to non-limiting embodiments wherein the implant is optionally made of at least one or more biodegradable, degradable and/or perishable parts and or portions.

Optionally the implant may according to optional embodiment of the present invention may be further provided with a coating, for example including but not limited to a therapeutic agent, drug, a medicaments similar to drug-eluting materials or the like.

Optionally the implant according to non-limiting embodiment wherein the implant lumen may be provided with a compressible biocompatible substance such as absorbable gelatin sponge, or other absorbable or non-absorbable biocompatible substance, or the like.

Optionally the implant according to non limiting embodiments of the present invention may be introduced through at least one or both of the cervical, lumbar, thoracic or sacral vertebral pedicles. Optionally the implant may be utilized for height restoration for example following vertebral body compression (possibly from a fracture) and/or in order to serve as a scaffold or base provided for strengthening such a vertebra before injecting hardening material into the vertebra, for example such as bone cement, hydroxyapatite or similar material that is injected in a fluid form and hardens in place.
Optionally the implant may according non limiting optional embodiment of the present invention may be provided from materials for example including but not limited to nitinol, stainless steel 316, memory shape polymers, beryllium copper alloys, cobalt-chromium-molybdenum alloy, cobalt chrome alloy, biological tissue, any combination thereof, or the like.
Optionally the implant may according non limiting optional embodiment of the present invention may be provided in a crimped and/or minimal profile prior to implantation and may be deployed to its full sized within the implantation site.
An optional embodiment of the present invention provides for a method for repairing a tear in an annulus fibrosus of a spinal disc wherein the tear comprises a distal surface, medial surface and a proximal surface; by implanting an implant according to optional non limiting embodiments of the present invention within the tear the method comprising:
associating an implant with a delivery system and accessing target site and clearing an implant delivery path toward the tear; and urging the distal portion of the implant into the distal surface of the tear; and urge the medial portion of the implant into the medial surface of the tear; and controllably anchor the implant distal portion along the distal surface; and controllably anchor the implant medial portion along the medial surface; and urge the proximal portion of the implant into the proximal surface of the tear and controllably anchor the implant proximal portion along the proximal surface.
Optionally the implant may be further associated with an optional membrane with the proximal portion of said implant along the proximal surface of said tear.
An optional embodiment of a non limiting embodiment of the implant according to the present invention where the implant may provide for a unidirectional valve for example for injecting hardening material, or optionally by preventing the material from leaking into the spinal canal/channel. Leakage of hardening material is the main potential complication resulting from such injections and is prevalent in up to 40% of instances.

An optional embodiment of the present invention provides for implanting the implant through a standard surgical incision following an accepted standard procedure for a partial excision of a herniated inter-vertebral disc, according to standard procedures for an operation of this kind. The implant may be implanted following a standard discectomy (open), a discectomy using microsurgical techniques (microscopic discectomy), a minimally invasive discectomy, or in place of a discectomy by inserting the implant through the tear in the annulus fibrosus without performing a discectomy, as is the case with degenerated inter-vertebral discs. In addition, the implant can be implanted using various methods such as those employed for a inter-vertebral discectomy in the various regions of the spine, including posterior approaches, anterior approach, paravertebral approach (WILTSE approach), minimally invasive approach and more. The implant will seal the aperture or defective area in the annulus fibrosus of the disc with its round, oblong or irregular shape

An optional embodiment of the present invention provides for a unidirectional permeability of the implant that may optionally be a key component for use in situations where liquid build up needs to be drained on a constant basis in different cavities in the body (for example, in the pleural space where excess fluid accumulates or in the abdominal cavity where fluid accumulates, or to allow drainage of cerebral spinal fluid (CSF) for people suffering from acute elevation of intracranial pressure due to various causes—where the drainage of fluids in each of these (and many other) cases is needed).

Unless otherwise defined, all technical and scientific terms used herein have the same meaning as commonly understood by one of ordinary skill in the art to which this invention belongs. The materials, methods, and examples provided herein are illustrative only and not intended to be limiting. Implementation of the method and system of the present invention involves performing or completing certain selected tasks or steps manually, automatically, or a combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

IN the Drawings:

FIG. 1A-B are a schematic illustration of an intervertebral disc, where FIG. 1A depicts a healthy disc, while FIG. 1B depicts a disc comprising an annular fissure tear, that may optionally and preferably by sealed with the implant and method of implant delivery according to the present invention.

FIG. 2A-C show optional views of a schematic illustration of an optional non limiting embodiment of the implant according to an optional non-limiting embodiment of the present invention.

FIG. 3A-D show optional views of a schematic illustration of an optional non limiting embodiment of the implant according to an optional non-limiting embodiment of the present invention.

FIG. 4A-C show optional views of a schematic illustration of an optional non limiting embodiment of the implant according to an optional non-limiting embodiment of the present invention.

FIG. 5A-C show optional views of a schematic illustration of an optional non limiting embodiment of the implant according to an optional non-limiting embodiment of the present invention.

FIG. 6A-D show optional views of a schematic illustration of an optional non limiting embodiment of the implant according to an optional non-limiting embodiment of the present invention.

FIG. 7A-F show optional views of a schematic illustration of an optional non limiting embodiment of the implant according to an optional non-limiting embodiment of the present invention.

FIG. 8A-C show optional views of a schematic illustration of an optional non limiting embodiment of the implant according to an optional non-limiting embodiment of the present invention.

FIG. 9A-C show optional views of a schematic illustration of an optional non limiting embodiment of the implant according to an optional non-limiting embodiment of the present invention.

FIG. 10A-C show optional views of a schematic illustration of an optional non limiting embodiment of the double lumen implant according to an optional non-limiting embodiment of the present invention.

FIG. 11A-D show optional views of a schematic illustration of an optional non limiting embodiment of a double lumen implant according to an optional non-limiting embodiment of the present invention.

FIG. 12 is an exemplary method for the delivery of an optional implant for sealing a spinal annular fissure tear according an optional embodiment of the present invention.

FIG. 13A-C provide schematic illustrations of optional implant according to the present invention disposed and implanted within a delivery site within an inter-vertebral disc.

FIG. 14A-B show optional views of a schematic illustration of an optional non limiting embodiment of a double lumen X shaped implant 195 according to an optional non-limiting embodiment of the present invention.

FIG. 15A-B show optional views of a schematic illustration of an optional non limiting embodiment of the present invention wherein bars are utilized across at least two spinal structures for example a disc and/or vertebrae.

### DESCRIPTION OF THE PREFERRED EMBODIMENTS

The present invention relates to an implantable device and a method for delivering the implant for the repair and closure of annular defects and in particular, to such a device and method in which spinal annular defects are repaired with an elastic-plastic implant adept to securely seal an annular defect while providing a sufficiently compliant implant that adapts to bodily movements.

The principles and operation of the present invention may be better understood with reference to the drawings and the accompanying description.

The following reference labels are used throughout the drawings to refer to objects having similar function, meaning, role, or objective.
5 inter-vertebral disc;
15 annular fissure tear, implantation site;
20 annulus fibrosus;
22 nucleus pulposus;
24 distal surface of annular fissure tear;
26 proximal surface of annular fissure tear;
28 luminal and/or medial surface of annular fissure tear;
100 implant;
102 anchors;
102d distal anchors;
102p proximal anchors;
104 implant body;
106 distal portion;
108 proximal portion;
110 implant;
112 anchors;
112d distal anchors;
112p proximal anchors;
114 implant body;
116 distal portion;
118 proximal portion;
120 implant;
122 anchors;
122d distal anchors;
122p proximal anchors;
124 implant body;
124a,b implant body portion;
124c implant body central axis;
124t implant body shaping tool;
126 distal portion;
128 proximal portion;
130 implant;
132 anchors;
132d distal anchors;
134 implant body;
136 distal portion;
138 proximal portion;
138m proximal portion membrane;
140 implant;
141 elongated rods;
141c central portion;
141d distal flanking portion;
141p proximal flanking portion;
142 anchors;
142d distal anchors;
142p proximal anchors;
144 implant body;
146 distal portion;
148 proximal portion;
150 implant;
152 anchors;
152d distal anchors;
152p proximal anchors;
154 implant body;
154t implant body threading;
155 sealing membrane;
156 distal portion;
158 proximal portion;
160 implant;
162 curved wire frame members;
164 spherical wire frame implant body;
168 proximal portion;
170 implant;
172 curved wire frame members;
174 dual spherical wire frame implant body;
176 distal portion;
178 proximal portion;
180 implant;
182 first support structure;
182p proximal portion of first support structure;
182d distal portion of first support structure;
182m medial portion of first support structure;
184 second support structure;
184p proximal portion of second support structure;
184d distal portion of second support structure;
184m medial portion of second support structure;
190 double lumen implant;
192 first support structure;
192p proximal portion of first support structure;
192d distal portion of first support structure;
192m medial portion of first support structure;
194 second support structure;
194a central portion of second support structure;
194b right arm portion of second support structure;
194c left arm portion of second support structure;
195 X shaped implant;
197 first support member;
199 second support member.

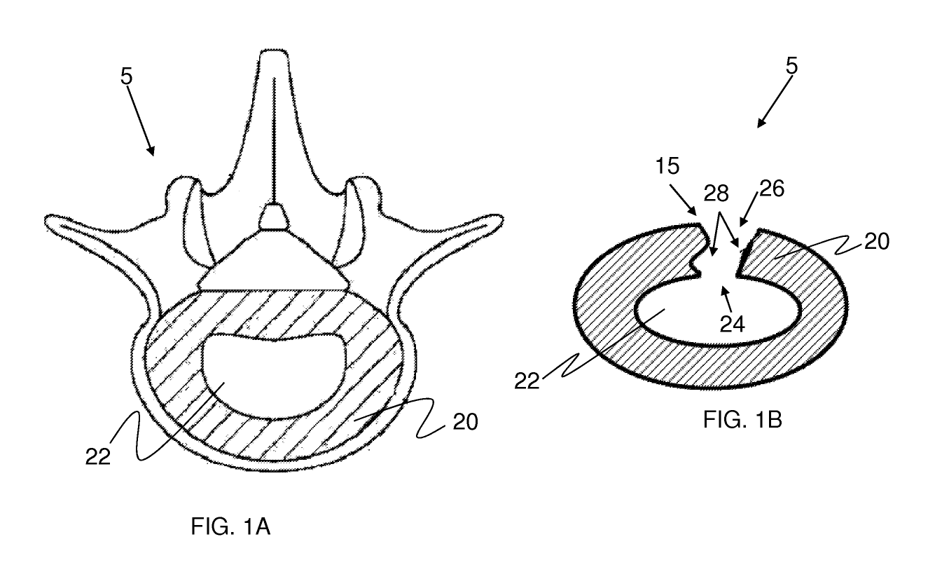

Referring now to the drawings, FIG. 1 shows a schematic illustration of a intervertebral disc 5 comprising an external fibrous ring structure called the annulus fibrosus 20 or annulus that maintains an internal viscous sponge-like structure called the nucleus puposus 22 or disc nucleus. As previously described, over time, as disc 5 deteriorates, an annular fissure tear 15 may form in annulus 20, as shown in FIG. 1B, which may lead to displacement or herniation of the disc nucleus 22 that would press onto neural tissue such as a nerve root and/or the dural sac itself (not shown). Herniation of nucleus 22 through tear 15 and onto neural tissue may lead to pain, discomfort, or neurological damage as previously described. Various methods have been devised and are used to correct herniated discs, where disk nucleus 22 displaces outside the confines of the annulus fibrosus and presses on neural tissue. These methods were previously described herein and are collectively termed discectomy, for they are aimed at removing, or excising, the herniated portion of the inter-vertebral disc.

Although some devices and implants have been introduced to seal the opening formed by annular tear, 15 such implants do not offer a device for sealing annular tear where the implant comprises dual properties where optionally preferably it is both sufficiently plastic and/or strong to maintain, and provide support for the portion of the spine wherein it is disposed. Most preferably the implant according to an optional embodiment of the present invention is further competent in absorbing the various pressures exerted on the spine. Optionally and preferably while the implant is adept at providing support it most preferably further provides for flexible and plastic support at the implantation site so as to provide for a full range of motion associated with the implantation site.

Optionally, a plurality of optional implants may be utilized according to the present invention to provide for both elastic and plastic support at the implantation site, 15 preferably by providing for at least one or more anchoring sites. Optionally, elastic and plastic support is provided by anchoring an optional implant according to the present invention in at least one location along the tear 15. More preferably anchoring of an optional implant according to the present invention is provided for in at least two or more locations along the tear 15. Optionally and preferably anchoring is provided for in at least three locations along the tear 15.

Optionally anchoring an implant according to the present invention along the annular tear 15 may be provided for in at least one location along annular tear 15, optionally and preferably in at least two locations and most preferably in at least three locations. Optionally anchoring location along annular tear 15 is chosen from the distal surface 24, proximal surface 26, or luminal surface 28, or the like along the path of annular tear 15.

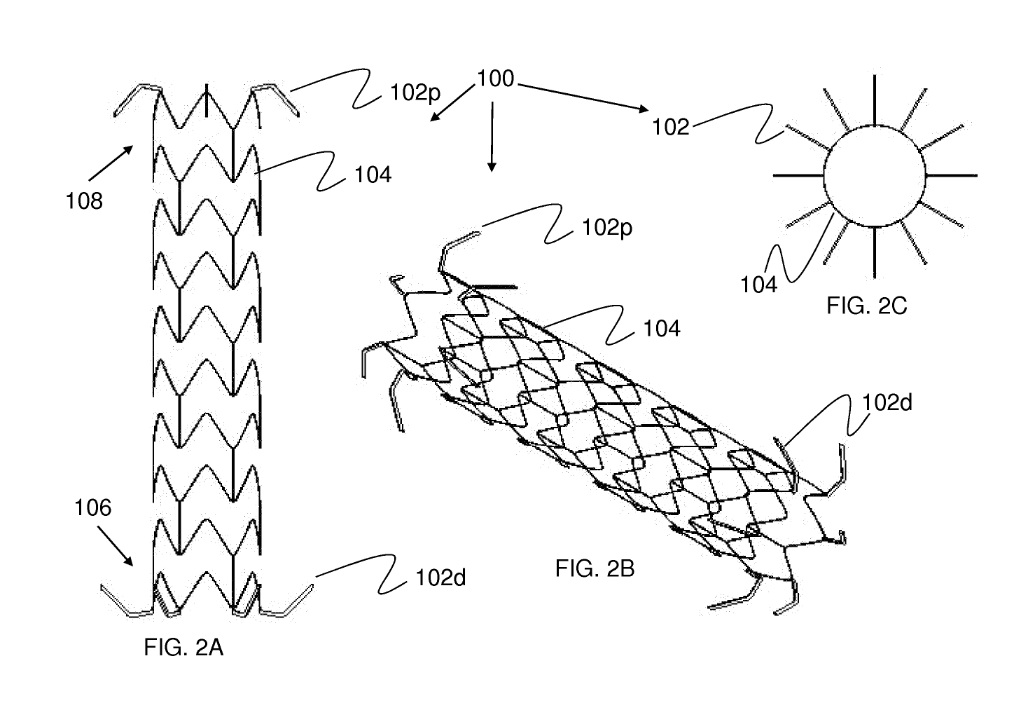

Optionally anchoring along the distal surface 24 provides for anchoring an optional implant according to an optional embodiment of the present invention as will be depicted in FIGS. 2-7 below, along the nucleus side or inner surface of annulus 20 of disc 5 most preferably without disturbing the nucleus 22, and preferably maintaining the native volume of the nucleus pulposus 22.

Optionally anchoring along the proximal surface 26 provides for anchoring an optional implant according to an optional embodiment of the present invention as will be depicted in FIGS. 2-7 below, along the outer surface of annulus 20 of disc 5.

Optionally anchoring along the medial and/or luminal surface 28 provides for anchoring an optional implant according to an optional embodiment of the present invention as will be depicted in FIGS. 2-7 below, along the length of tear 15 formed in disc 5.

FIGS. 2-7 provide optional depiction of a plurality of optional implantation device 100, 110, 120, 130, 140, 150 for the sealing of an annular tear 15 according to optional embodiments of the present invention. Most preferably implantation devices 100, 110, 120, 130, 140, 150 may be adapted to provide for at one, more preferably at least two and most preferably at least three anchoring sites along tear 15. Most preferably anchoring may be provided with anchors 102, 112, 122, 132, 142, 152 along distal surface 24, proximal surface 26 or medial surface 28.

Optionally anchors 102, 112, 122, 132, 142, 152 may for example be provided in the form of rod like structures and/or threading or the like. Optionally rod like anchors may be controllably shaped in accordance with a plurality of optional parameters for example including but not limited to shape of annular tear 15, available anchoring space, physician assessment, or the like.

Optionally and preferably deployment of anchors is provided by a controllable double back technique such for example a distal anchor 102d is disposed distally at the distal surface 24 and is bent to double back proximally toward therein preferably forming a hook like anchor providing for support of the implant within the annular tear 15 defining the implantation site.

Optionally and preferably implant 100, 110, 120, 130, 140, 150 provides for dual elastic and plastic support within annular tear 15 forming the implantation site, in providing an implant body 104, 114, 124, 134, 144, 154 that may be anchored to the luminal and/or medial surface 28 of annular tear 15 providing for the plastic supportive qualities. Optionally medial anchoring along implant body 104, 114, 124, 134, 144, 154 may be provided in the form of hook like rods, threading or the like. Optionally and preferably the flexible and/or elastic properties are provided for with controllable parameters associated with the implant body for example including but not limited to contour, shape, size, dimensions, materials or the like. Optionally implant body may be adjusted to securely fit and/or associate with the shape and/or contour of annular tear 15.

Optionally implant 100, 110, 120, 130, 140, 150 may be disposed with at least one or more membrane preferably along at least one or more of the proximal, distal or medial (body) portion of the implant. Optionally the membrane may be provided as a selectively permeable member providing for sealing annular tear 15 along at least one of its distal, proximal or medial surfaces. Optionally a membrane may further provide for delivering medicament, drugs, hormones, therapeutic agent or the like to the implantation site 15.

Referring now to FIG. 2A-C, providing an option non limiting embodiment of the present invention of implant 100 in the form of a stent-like implant comprising a plurality of anchors 102 and implant body 104. FIG. 2A-B provide perspective views of implant 100 while FIG. 2C provides a cross-section of implant 100.

Optionally anchors 102 may be disposed along the distal portion 106, proximal portion 108 or along the medial body portion 104. Optionally the number of anchors disposed and/or deployed on any one side is controllable. Optionally the number of anchors 102 utilized is dependent on the size and the like parameters of the implantation site 15. For example an annular tear 15 may be such that the distal surface 24 is wider than the proximal surface accordingly a larger number of distal anchors 102d would be disposed along the distal surface 24 to provide for sufficient anchoring while proximal anchors 102p may be disposed along the proximal surface 26.
Optionally and preferably proximal anchors 102p are deployed proximally and thereafter bent, folded, manipulated, displaced distally with the implantation site forming a hook like anchor within at the proximal surface 26. Most preferably the deployment of anchors 102p is controllable during implantation and may be controlled by a practitioner for example the number anchors utilized, the direction of anchoring, the degree of folding, shape of anchor or the like parameters may be controlled.
Optionally and preferably distal anchors 102d are deployed distally and thereafter bent, folder, manipulated, displaced proximally with the implantation site forming a hook like anchor within at the proximal surface 24. Most preferably the deployment of anchors 102d is controllable during implantation and may be controlled by a practitioner—for example, the number anchors utilized, the direction of anchoring, the degree of folding, shape of anchor or the like parameters may be controlled.

Optionally implant medial portion and/or body 104 is depicted in the form of a stent-like wire mesh may be provided in optional forms where body 104 may be shaped to conform with the shape of the implantation site 15. Optionally the shape of implant body 104 may be controlled by a practitioner during deployment optionally with an auxiliary device for example in the form of a balloon, semi-compliant balloon or the like to radially affix and associate implant body 104 with medial surface 28 of implantation site 15, therein optionally and preferably conforming implant body 104 with the shape of implantation site 15. Optionally implant body 104 may be provided from optional materials for example including but not limited to NiTinol, medical grade stainless steel 316 or the like compliant and resilient materials that may provide for the dual nature of implant 100 elastic and plastic properties.

Optionally the number of anchors utilized and/or disposed along any portion of implant 100 may be individually controlled.

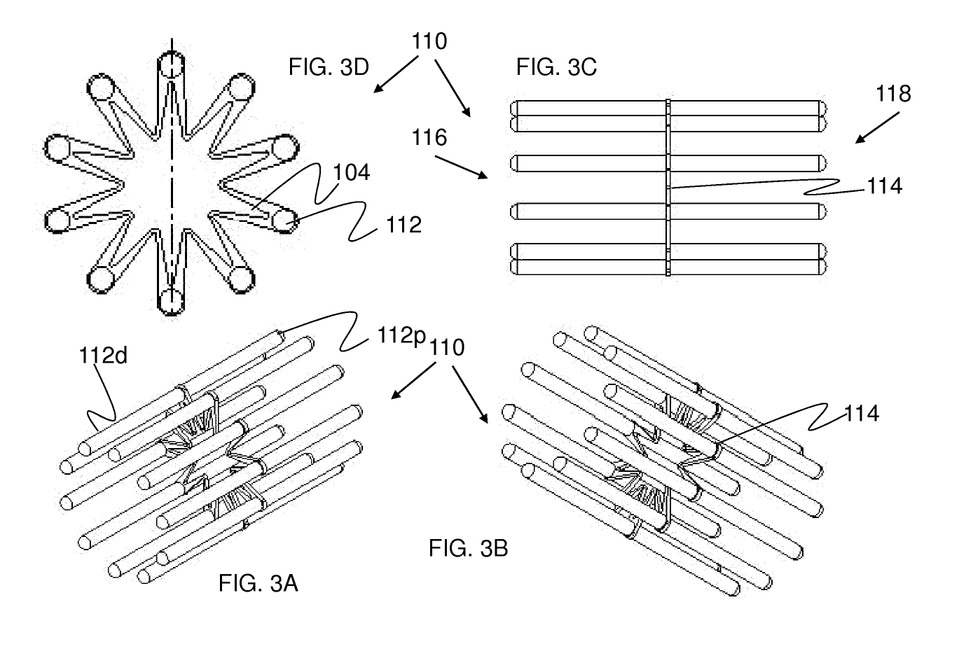

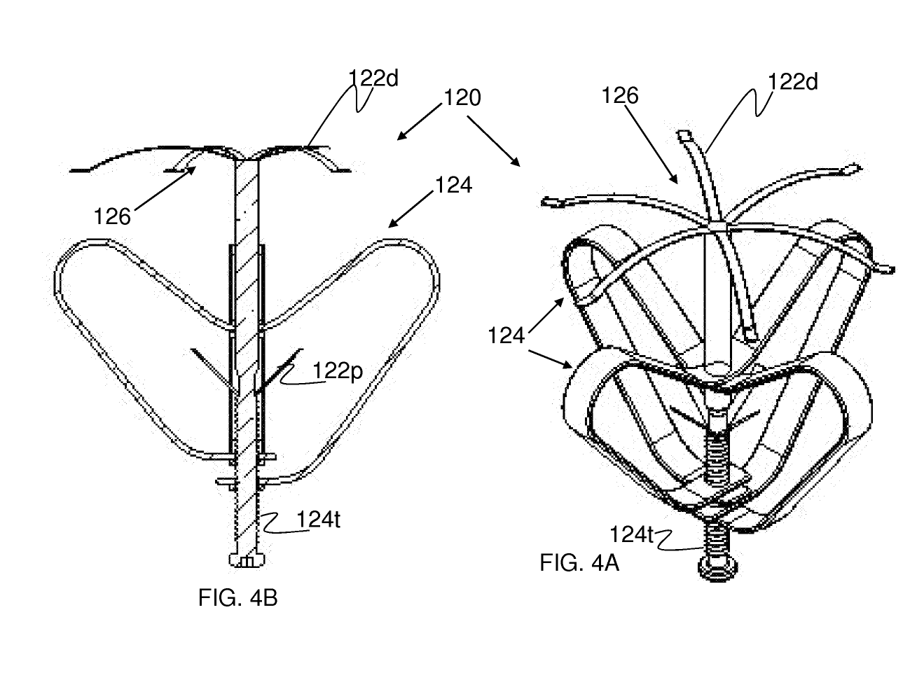

Referring now to FIG. 3A-D, providing an option non limiting embodiment of the present invention of implant 110 in the form of a rink-like implant comprising a plurality of anchors 112 and implant body 114. FIG. 3A-B provide perspective views of implant 110, FIG. 3D provides a cross-section of implant 110 and FIG. 3C provides a side view of implant 110. Optionally implant body 114 is depicted in a ring like structure that may be controllably adjusted to fit implantation site 15. Optionally proximal portion 118 may be provided with anchors 112p while distal portion 116 may be provided with anchors 112d. Optionally anchors 112 may be provided in the form of pressure anchors that may be pressed along the medial portion 28 of implantation site 15 therein anchoring with the use of pressure. Optionally implant 110 may be deployed with the aid of an expandable balloon to securely associate with implantation site while separately controlling the shape of the implant body 114, proximal portion 118 and distal portion 116.

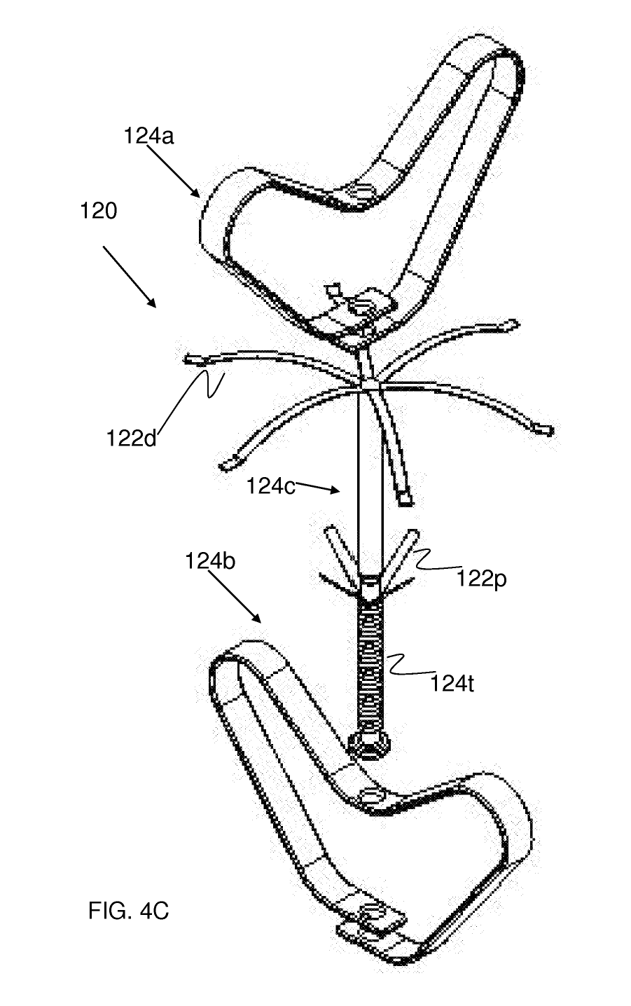

Referring now to FIG. 4A-C, providing an option non limiting embodiment of the present invention of implant 120 comprising a plurality of anchors 122 and implant body 124. FIG. 4A provides a perspective view, FIG. 4B provides a side view while FIG. 4C provides an exploded view of implant 120. Optionally implant body 124 comprise at least three or more portions 124a, 124b, 124c wherein portions 124a and 124b may be shaped to fit the medial portion of implantation site 15, about a central axis 124c. Optionally the shape of implant 120 may be provided by manipulating an implant body shaping tool 124t for example in the form of a threadable screw that may be adjusted to control the shape of body 124 by manipulating with corresponding threading disposed intraluminally (not shown) with body 124c.

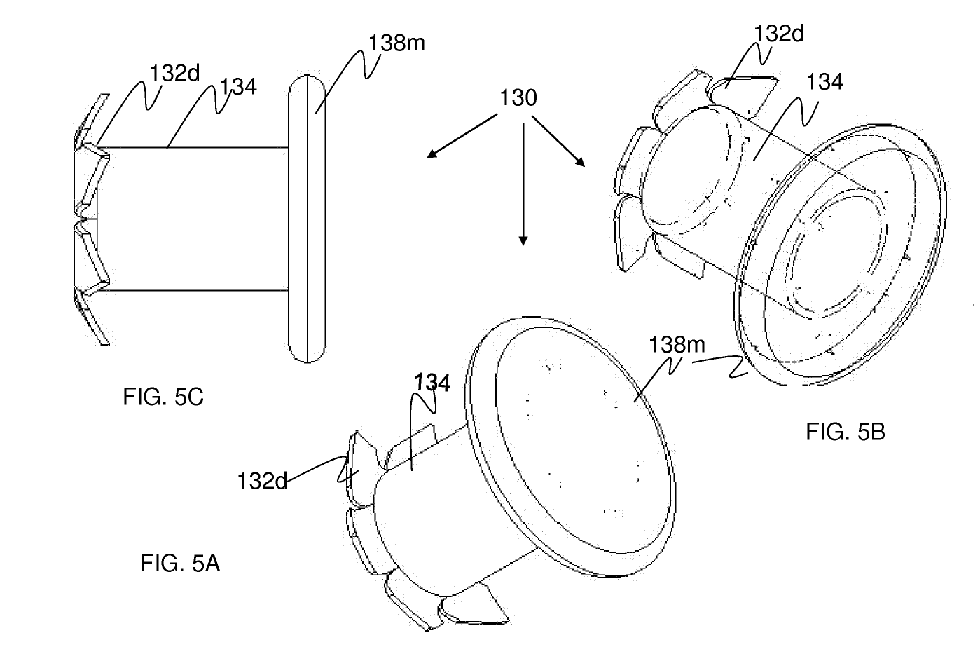

Referring now to FIG. 5A-C, providing an option non limiting embodiment of the present invention of implant 130 provided in the form of a tubular implant similar to implant 100 depicted in FIG. 2. Implant 130 comprises a plurality of distal anchors 132d, implant body 134 and a proximal portion membrane 138m. FIG. 5A-B provides a perspective views while FIG. 5C provides a side view of implant 130.

Optionally implant 130 comprises anchors only at its distal portion 136 while proximally a selectively permeable member 138m provides for anchoring and support while optionally and preferably preventing nucleus fluid 22 from flowing proximally through tear 15. Optionally implant body 134 provides for support along the medial portion 28 of tear 15. Optionally and preferably the shape of implant body 134 may be controlled by a practitioner during deployment to securely and flexibly associate within implantation site 15. Optionally implant 130 may be provided with proximal anchors (not shown) that may be associated with proximal membrane 138m. Optionally implant 130 may be provided with distal anchors (not shown) that may be associated with a selectively permeable membrane (not shown).

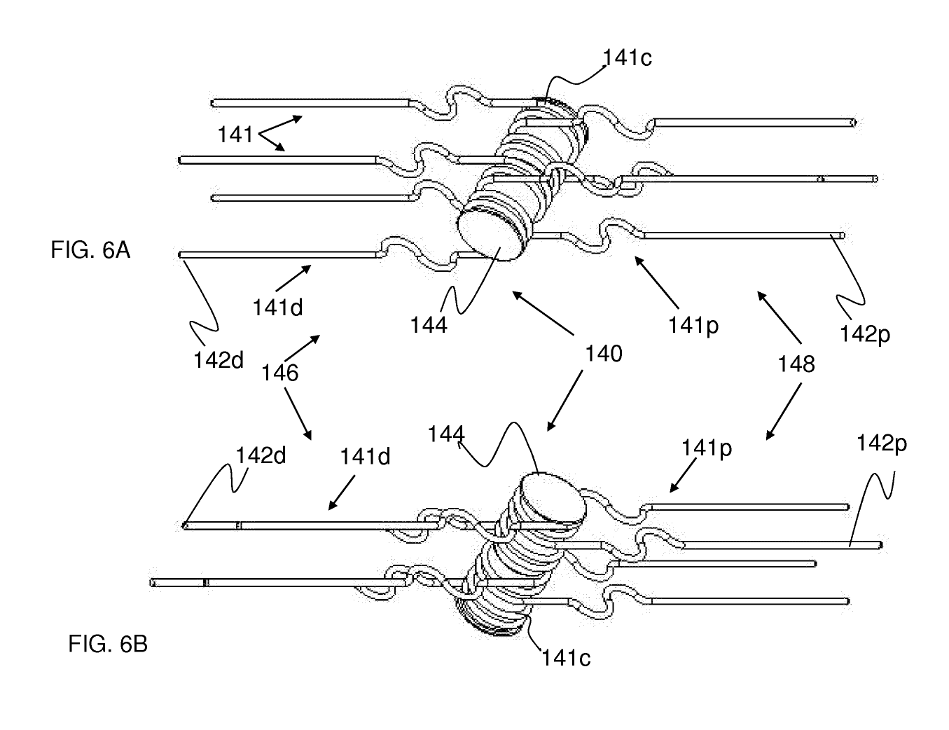

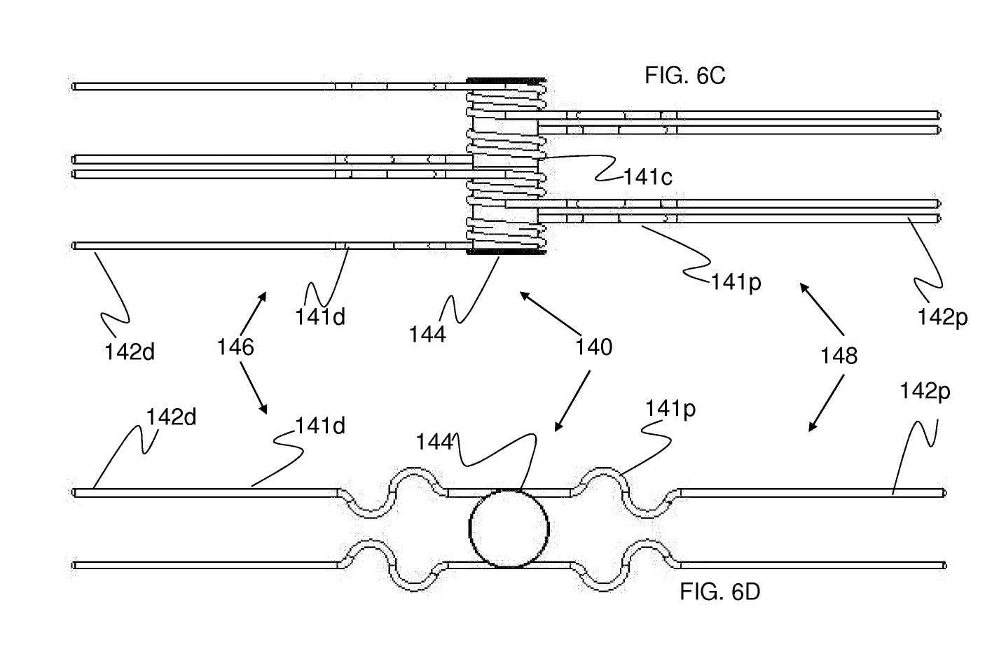

Referring now to FIG. 6A-D, providing an optional non limiting embodiment of the present invention of an implant 140. FIG. 6A-B show perspective views while FIG. 6C-D show different side views of implant 140. Implant 140 comprises a substantially tubular body 144 optionally and preferably forming a scaffold along which at least one and more preferably a plurality of elongated rods 141 may be associated with. Elongated rods 141 are optionally associated with body 144 by circumferentially looping rods 141 about the axis of body 144 and along the length of the external surface of body 144 therein forming at least two or more portions 141d, 141p flanking body 144. Once associated with flanking body 144 rods 141 preferably comprise a distal flanking portion 141d, a central portion 141c and a proximal flanking portion 141p.

Optionally flanking portions 141p and 141d may be provided in a plurality of optional shapes and/or contours for example including portions that are substantially linear, substantially curved, straight, curved, angled, looped, sigmoid or the like. For example flanking portions 141d and 141p are schematically depicted in FIG. 6A-D as having a sigmoid contour. Optionally the shape of flanking portions 141p and 141d may be individually controlled and/or manipulated to securely associate within the implantation site 15 while maintain the overall flexibility of structures about the implantation site without compromising the seal formed about the annular tear 15.

Preferably the distal and proximal ends of rod 141 provide for anchoring implant 140 within the implantation site 15, therein forming anchors 142, distal anchors 142d and proximal anchors 142p. As previously described anchors 142 may be controllably placed and shaped within the implantation site. For example, a proximal anchor 142p may be deployed proximally and then optionally displaced distally to form a hook like contour adept for anchoring within implantation site 15. For example distal anchor 142d may be deployed distally and then optionally displaced proximally forming a hook like contour within the implantation site adept for anchoring within implantation site 15.

Optionally the shape of central portion 141c may be controlled during implantation to best fit the implantation site. Optionally central portion 141c may be shaped with an auxiliary device for example including but not limited to a semi-compliant balloon, balloon, or the like.

Optionally body 144 and central portion 141c may be remain associated during and following implantation. Optionally the shapes of both body 144 and central portion 141c may be simultaneously controlled during implantation to best fit implantation site 15. Optionally body 144 may be shaped with an auxiliary device for example including but not limited to a semi-compliant balloon, balloon, or the like, that may be expanded within the implantation site optionally and preferably to assume the implantation site shape.

Optionally tubular body 144 may optionally be evacuated from the implantation site 15 optionally following the anchoring of rods 142, 142d, 142p.

Optionally body 144 may be provided in a stent-like structure or mesh and made of materials for example including but not limited nitinol, stainless steel 316, memory shape polymers, beryllium copper alloys, cobalt-chromium-molybdenum alloy, cobalt chrome alloy, or the like as is know in the art.

Optionally individual rods 141 may be provided in the form of a continuous structure made of a single piece of materials. Optionally individual rods 141 may be provided in compound form for example including but not limited to a mesh, wire net, braid or the like. Optionally rods 141 may be provide from a plurality of optional materials for example including but not limited to polymers, nitinol, stainless steel 316, memory shape polymers, beryllium copper alloys, cobalt-chromium-molybdenum alloy, cobalt chrome alloy, titanium allow or the like as is know in the art.

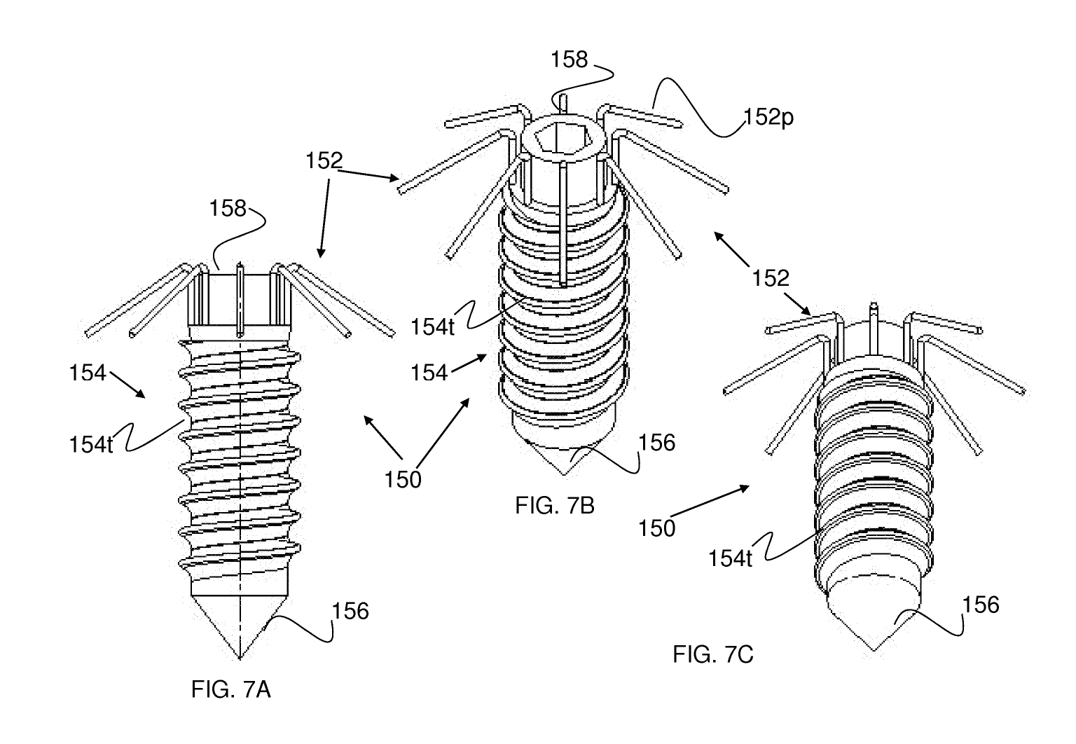

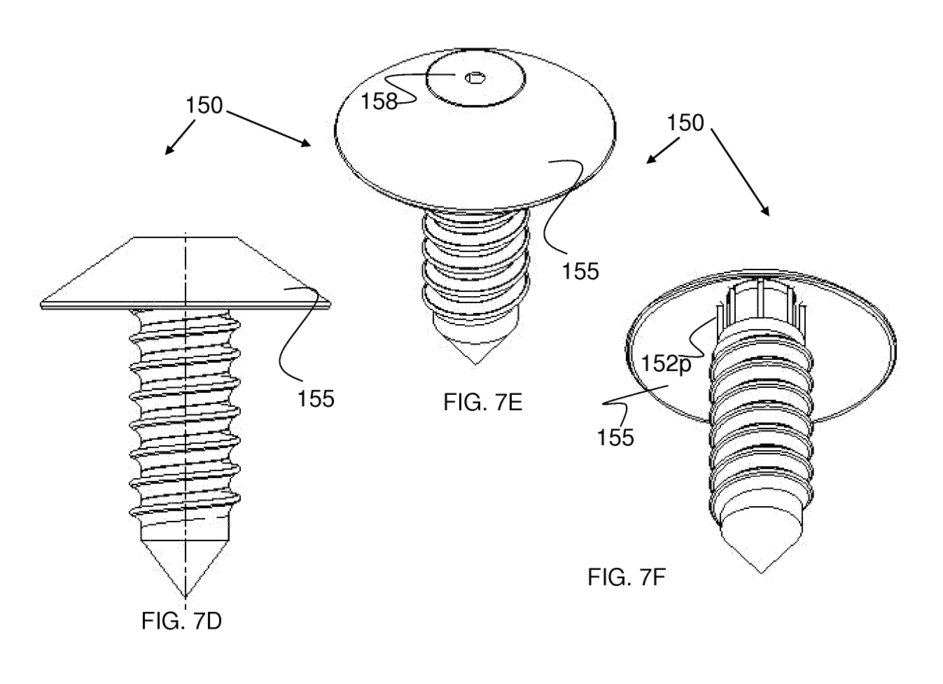

Referring now to FIG. 7A-F, providing an optional non limiting embodiment of the present invention of an implant 150 provided in the form of a screw body 154 comprising a plurality of anchors 152. FIG. 7A-C show varying perspective views of implant 150. Implant body 154 provided in the form of screw preferably comprises threading 154t adept for anchoring implant 150 within implantation site 15 and most preferably along its medial portion 28. Implant 150 preferably comprises a distal portion 156 and a proximal portion 158.

Preferably at least one of distal portion 156 and proximal portion 158 are provided with anchors 152. Most preferably proximal portion 158 comprise anchors 152p as shown adept for anchoring implant 150 within implantation site 15 along proximal surface 26. Optionally anchors 152 are disposed along both distal portion 156 and proximal portion 158. Optionally anchors may be further disposed along the implant body 154, for example extruding from threading 154t, not shown. Optionally implant 150 may be provided with proximal anchors (not shown) that may be associated with proximal portion 158.

Optionally implant 150 may be delivered and implanted into implantation site 15 with at least one or more auxiliary tools for example including a drill, screw driver, key or the like tools for securely associating implant 150 with in implantation site 15 where threading 154t may associate with the medial surface 28 of the implantation site 15. Optionally implant 150 may be manipulated, for example by threading, into the implant site 15 with an auxiliary device (not shown) through a its proximal end 158. Optionally proximal end 158 may be shaped to provide for associating with an auxiliary tool for example a drill, driver, key or the like, to facilitate implantation.

Optionally implant body 154 may for example be provided in optional formations for example including but not limited to a mesh-like (not shown), stent like (not shown), solid, hollow, semi-hollow (not shown), or the like formation. Optionally implant body provided in a hollow formation may be further anchored within implantation site 15 through a radial expanding auxiliary tool for example including but not limited to semi-compliant balloon, balloon or the like, that is optionally introduced through an opening in the proximal end 158.

Optionally implant 150 may be associated with at least one or more membrane 155 for sealing annular tear 15 and/or preventing any future herniation of nucleus pulposus 22, as schematically depicted in FIGS. 7D-F.

Optionally and preferably member 155 may be disposed along the any portion of implant 150 more preferably along the distal portion 156 or proximal portion 158, and most preferably proximal portion 158. Optionally member 155 may be associated with at least a portion of proximal anchors 152p, as schematically shown in FIG. 7D-F.

Optionally the membrane 155 may be semi-permeable, selectively permeable, permeable, or the like. Optionally membrane 155 facilitates delivery of a medicament, drug and/or therapeutic agent to the implantation site 15. For example, membrane 155 may be coated with a therapeutic agent to facilitate closure and/or healing of annular tear 15. Optionally and preferably member 155 provides for sealing annular tear 15 along at least the proximal surface 26, most preferably provided for preventing loss of nucleus pulposus 22f.

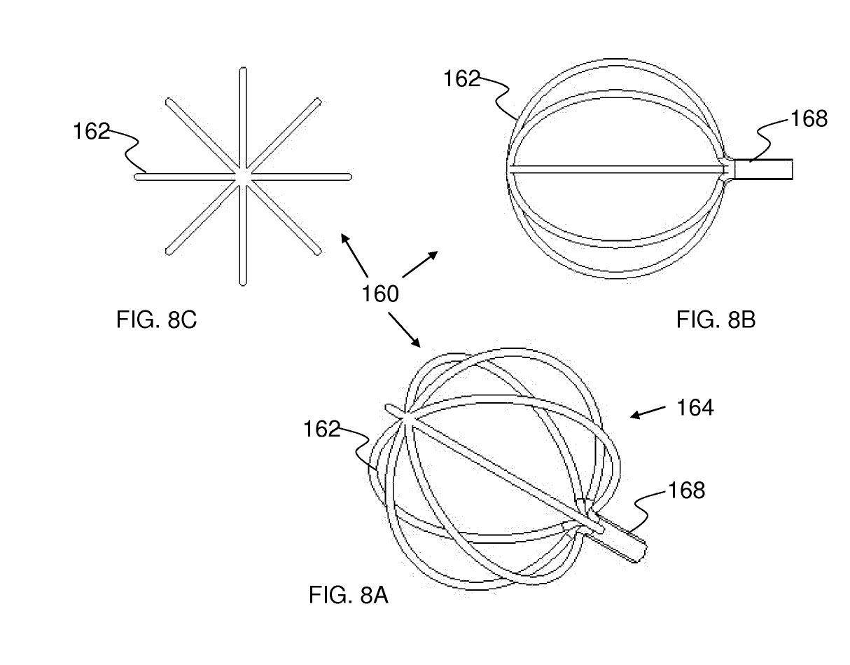

Referring now to FIG. 8A-C, providing an optional non limiting embodiment of the present invention of an implant 160 provided in the form of a spherical wire frame body 164 comprising a plurality of intermeshed curved wire frame members 162, optionally in the form elongated cylindrical rods having an optional shape and surface for example including but not limited to curved, coiled, bowed, curled arcs, spheres, circular, sigmoid or the like. FIG. 8A-B shows perspective views of implant 160 while FIG. 8C provides a cross sectional view. Preferably implant 164 further comprises a proximal member 168 that may optionally be removed partially following implantation. Optionally proximal member 168 provides a proximal seal disposed along proximal surface 26 of implantation site 15. Optionally implant 160 may be further associated with a membrane (not shown) as previously described.

Optionally implant 160 may be placed within implantation site 15 in a crimped and/or minimized state and controllably expanded to fill and seal annular tear 15, wherein implant 160 is adapted to securely and flexibly fit implantation site 15. Optionally individual curved wire frame member 162 may be controllably deployed within implantation site 15. Optionally implant 162 is radially expanded with an auxiliary tool for example including but not limited to a semi-compliant balloon, balloon, balloon catheter or the like.

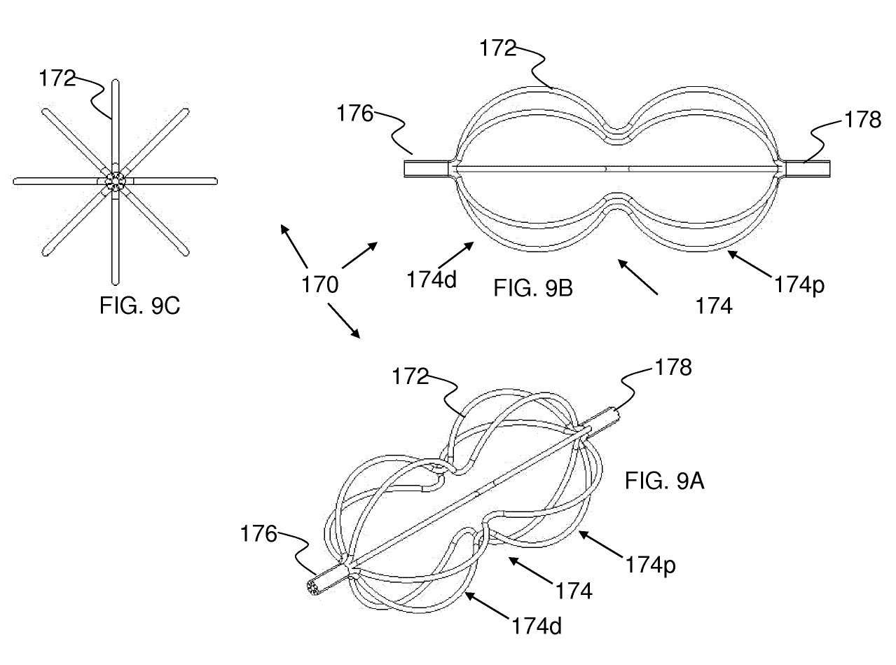

Referring now to FIG. 9A-C, providing an optional non limiting embodiment of the present invention of an implant 170 provided in the form of a dual spherical wire frame body 174 comprising a plurality of intermeshed curved wire frame members 172, optionally in the form elongated cylindrical rods having an optional shape and surface for example including but not limited to curved, coiled, bowed, curled arcs, spheres, circular, sigmoid or the like. FIG. 9A-B shows perspective views of implant 170 while FIG. 9C provides a cross sectional view. Dual spherical wire frame body 174 preferably comprises a distal spherical portion 174d and a proximal spherical portion 174p.

Preferably implant 170 further comprises a proximal member 178 and a distal member 176 that may optionally be partially removed following implantation. Optionally proximal member 178 and/or distal member 176 may provide a proximal seal disposed along proximal surface 26 and/or distal surface 24 of implantation site 15. Optionally implant 170 may be further associated with a membrane (not shown) for example along curved wire frame members 172.

Optionally implant 170 may be placed within implantation site 15 in a crimped and/or minimized state and controllably expanded to fill and seal annular tear 15, wherein implant 170 is adapted to securely and flexibly fit implantation site 15. Optionally individual curved wire frame member 172 may be controllably expanded within implantation site 15 to form a better fit. Optionally implant 172 is radially expanded with an auxiliary tool for example including but not limited to a semi-compliant balloon, balloon, balloon catheter or the like. Optionally individual portions of implant body for example distal portion 174d and/or proximal 174p may be individually expanded within implantation site 15 therein conforming implant 170 to the shape of annular tear 15.

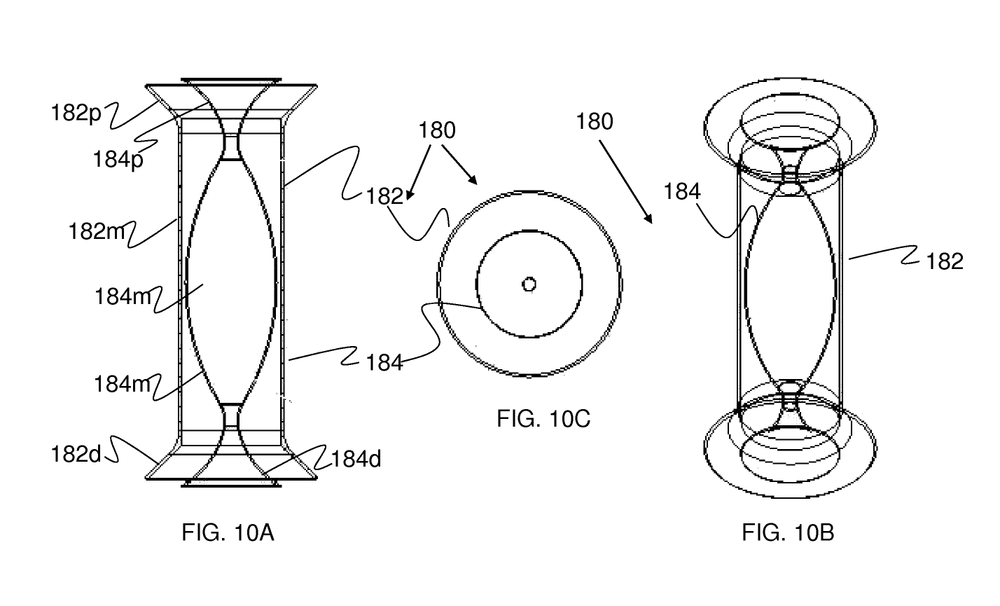

Referring now to FIG. 10A-C showing a schematic illustration of an optional non limiting embodiment of implant 180 according to the present invention. FIGS. 10A-B provide a perspective view while FIG. 10C provides a cross-section view of implant 180. Implant 180 comprises at least two or more interconnected support structures provided for sealing and flexibly supporting an annular tear 15. Implant 180 preferably comprises a first support member 182 and a second member 184. Optionally second support member 184 is disposed within the lumen of first support member 182. Optionally first and second support members 182, 184 are concentrically associated with one another therein sharing a common diameter. Optionally each support member 182, 184 may be provided in a configuration for example including but not limited to stent-like, wire mesh, wire frame, polymer or the like. Optionally, the external surface of support member 182, 184 may further comprise anchors (not shown) as previously described.

Optionally first support member 182 may be provided in a substantially tubular shape comprising a proximal portion 182p, distal portion 182d, and a medial portion 182m. Medial portion 182m preferably comprises a substantially tubular shape comprising a uniform diameter. Distal portion 182d extends distally from medial portion 182m comprising a gradually increasing diameter, as shown. Proximal portion 182p extends proximally from medial portion 182m comprising a gradually increasing diameter, as shown. Optionally at least one of proximal portion 182p, and/or distal portion 182d and/or medial portion 182m comprise anchors (not shown) provided for securely and flexibly associating within implantation site 15. Optionally and preferably anchors may be disposed on both distal portion 182d and proximal portion 182p.

Optionally second support member 184 comprises a proximal portion 184p, distal portion 184d, and a medial portion 184m, optionally substantially forming a hourglass shape. Medial portion 184m preferably comprises a substantially oval, elliptical contour as shown, wherein the diameter gradually tapers near the proximal and distal end. Distal portion 184d extends distally from medial portion 184m comprising a substantially conical shape that is flush and continuous with medial portion 184m, comprising gradually increasing diameter, as shown. Proximal portion 184p extends proximally from medial portion 184m comprising a substantially conical shape that is flush and continuous with medial portion 184m, comprising a gradually increasing diameter, as shown. Optionally at least one of proximal portion 184p, and/or distal portion 184d and/or medial portion 184m comprise anchors (not shown) provided for securely and flexibly associating within implantation site 15. Optionally and preferably anchors may be disposed on both distal portion 184d and proximal portion 184p.

Optionally and preferably at least one or both implant support members 182 and/or 184 may be further associated with a membrane (not shown) as previously described and optionally applied before and/or after implantation of the implants within the desired implantation site.

Optionally and preferably each support members 182, 184 may be individually controlled and/or manipulated within implantation site 15. Optionally and preferably any portion and/or segment of support members for example 182m, 182p, 182d, 184m, 184d, 184p, may be individually controlled and/or manipulated within implantation site 15. Preferably individual control of implant 180 in its individual members provides for controllably placing implant 180 within the implantation site 15 to best fit its shape and/or contour required for effectively sealing site 15; while providing flexible and/or elastic-plastic mechanical support of site 15 and surrounding anatomical structures and/or tissue.

Optionally and preferably support members 182, 184 provide implant site 15 with flexible and/or elastic-plastic support adept at supporting the variable forces exerted on implantation site 15 and/or surrounding anatomical structures while simultaneously effectively sealing annular tear 15 and optionally limiting and/or preventing future herniation of nucleus pulposus 22.

Most preferably implant 180 is provided in a crimped and/or minimal profile prior to implantation and may be deployed to its full sized within implantation site 15 with optional auxiliary devices for example including but not limited to a delivery catheter, a dedicated delivery system, endoscope, guiding catheter, guide wire, balloon over a guide wire or the like.

Optionally the crimped and/or minimal profile of first and second member 182, 184 are independent of one another.

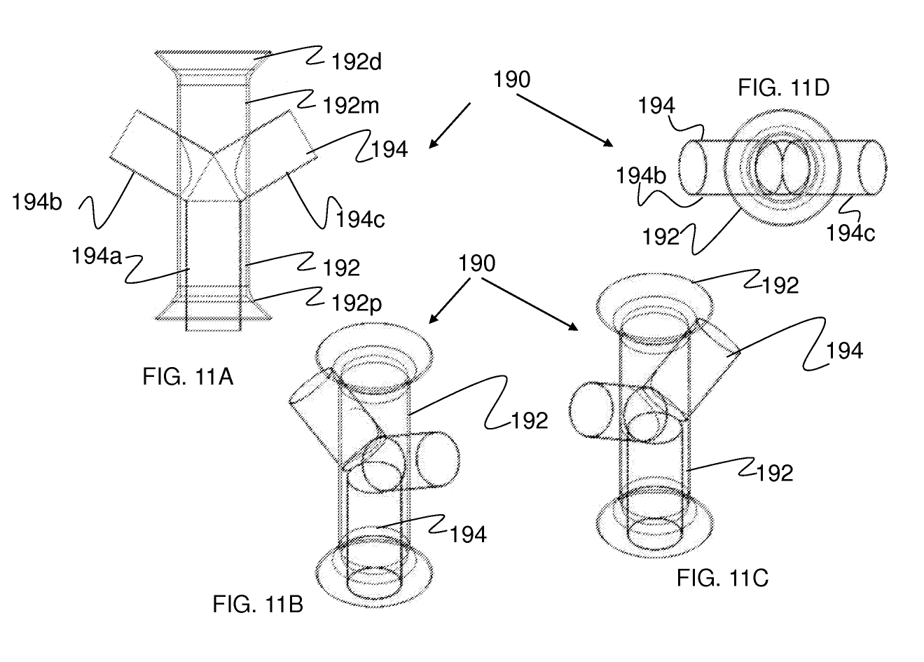

Referring now to FIG. 11A-D showing a schematic illustration of an optional non limiting embodiment of double lumen implant 190. FIGS. 11A-C provide perspective views while FIG. 11D provides a cross-section view of implant 190. Implant 190 comprises at least two or more interconnected support structures provided for sealing and flexibly supporting an annular tear 15. Implant 190 preferably comprises a first substantially tubular support member 192 and a second member 194. Optionally second support member 194 is associated with and at least partially disposed within first support member 192.

Optionally first support member 192 may be provided in a substantially tubular shape comprising a proximal portion 192p, distal portion 192d, and a medial portion 192m. Medial portion 192m preferably comprises a substantially tubular shape comprising a uniform diameter. Distal portion 192d extends distally from medial portion 192m comprising a gradually increasing diameter, as shown. Proximal portion 192p extends proximally from medial portion 192m comprising a gradually increasing diameter, as shown. Optionally at least one of proximal portion 192p, and/or distal portion 192d and/or medial portion 192m comprise anchors (not shown) provided for securely and flexibly associating within implantation site 15. Optionally and preferably anchors may be disposed on both distal portion 192d and proximal portion 192p.

Optionally and preferably support member 192 further comprises at least two or more recesses and/or openings (not shown) for receiving, accepting or otherwise associating with support member 194. Most preferably at least two recesses and/or opening are provided for along the medial portions 192m, optionally recess may be provided in the distal portion 192d and/or proximal portion 192p. Optionally and preferably such configuration further stabilizes and reinforces implant stabilization within implantation site 15.

Optionally second support member 194 may be provided as a ‘Y’ shaped support member substantially comprising central portion 194a, a right arm portion 194b, and left arm portion 194c. Optionally and preferably central portion 194a, a right arm portion 194b, and left arm portion 194c may be provided as a continuous single support structure. Most preferably each portion 194a, 194b, 194c, may be individually controlled and manipulated during implant delivery within implantation site 15. Optionally and preferably right arm portion 194b, and left arm portion 194c extend outside of the lumen of first support member 192, at an angle as shown.

Optionally central portion 194a, a right arm portion 194b, and left arm portion 194c may be provided as individual support members independent of one another while individually associated with first support member 192.

Most preferably implant 190 is provided in a crimped and/or minimal profile prior to implantation and may be deployed to its full sized within implantation site 15 with optional auxiliary devices for example including but not limited to a delivery catheter, a dedicated delivery system, endoscope, guiding catheter, guide wire, balloon over a guide wire or the like.

Optionally the crimped and/or minimal profile of first and second member 192, 194 are independent of one another.

Optionally and preferably support members 192, 194 provide implant site 15 with flexible and/or elastic-plastic support adept at supporting the variable forces exerted on implantation site 15 and/or surrounding anatomical structures while simultaneously effectively sealing annular tear 15 and optionally limiting and/or preventing future herniation of nucleus pulposus 22.

Optionally each support member 192, 194 may be provided in a configuration for example including but not limited to stent-like, wire mesh, wire frame, polymer or the like. Optionally, the external surface of support member 192, 194 may further comprise anchors (not shown) as previously described.

Optionally at least one of right arm portion 194b, and/or left arm portion 194c and/or central portion 194a comprise anchors (not shown) provided for securely and flexibly associating within implantation site 15. Optionally and preferably anchors may be disposed on both right arm portion 194b, and/or left arm portion 194c.

Optionally and preferably each support members 192, 194 may be individually controlled and/or manipulated within implantation site 15. Optionally and preferably any portion and/or segment of support members for example 192m, 192p, 192d, 194a, 194b, 194c, may be individually controlled and/or manipulated within implantation site 15. Preferably individual control of implant 190 in its individual members provides for controllably placing implant 190 within the implantation site 15 to best fit its shape and/or contour required for effectively sealing site 15; while providing flexible and/or elastic-plastic mechanical support of site 15 and surrounding anatomical structures and/or tissue.

Optionally and preferably implant 190 in any of its parts, for example support members 192, 194 may be further be further associated with a membrane (not shown) as previously described that is optionally applied before or after implantation of the implants within the desired implantation site. Optionally at least one or more constituents of implant 190 may comprise a selectively permeable membrane as previously described. Optionally a membrane (not shown) may be associated with at least one or more of 192, 192d, 192p, 192m, 194, 194a, 194b, 194c.

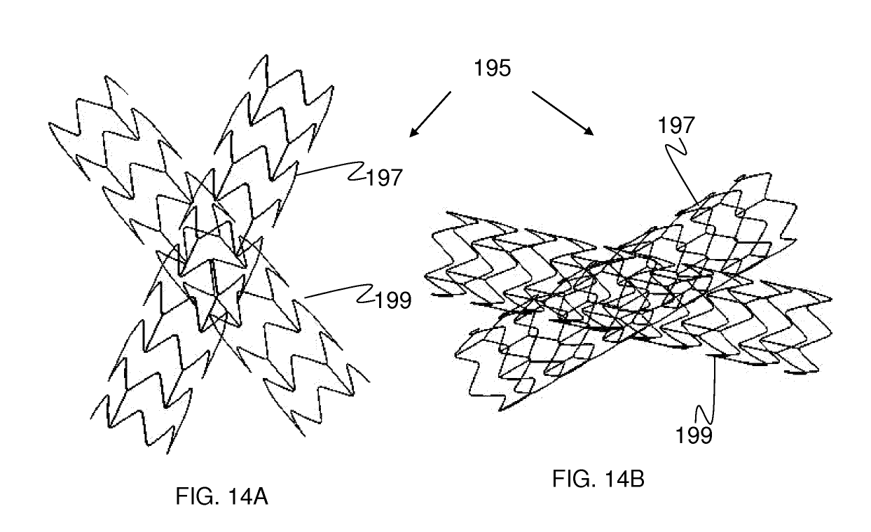

FIG. 14A-B show optional views of a schematic illustration of an optional non limiting embodiment of a double lumen ‘X’ shaped implant 195 according to an optional non-limiting embodiment of the present invention. Implant 195 comprises at least two or more interconnected support structures provided for sealing and flexibly supporting an annular tear 15. Implant 195 preferably comprises a first substantially tubular support member 197 and a second member 199. Optionally second support member 199 is associated with and at least partially disposed within first support member 197 forming a double lumen ‘X’ shape.

Optionally each support member 197, 199 may be provided in a configuration for example including but not limited to stent-like, wire mesh, wire frame, polymer or the like. Optionally, the external surface of support member 197, 199 may further comprise anchors (not shown) as previously described.

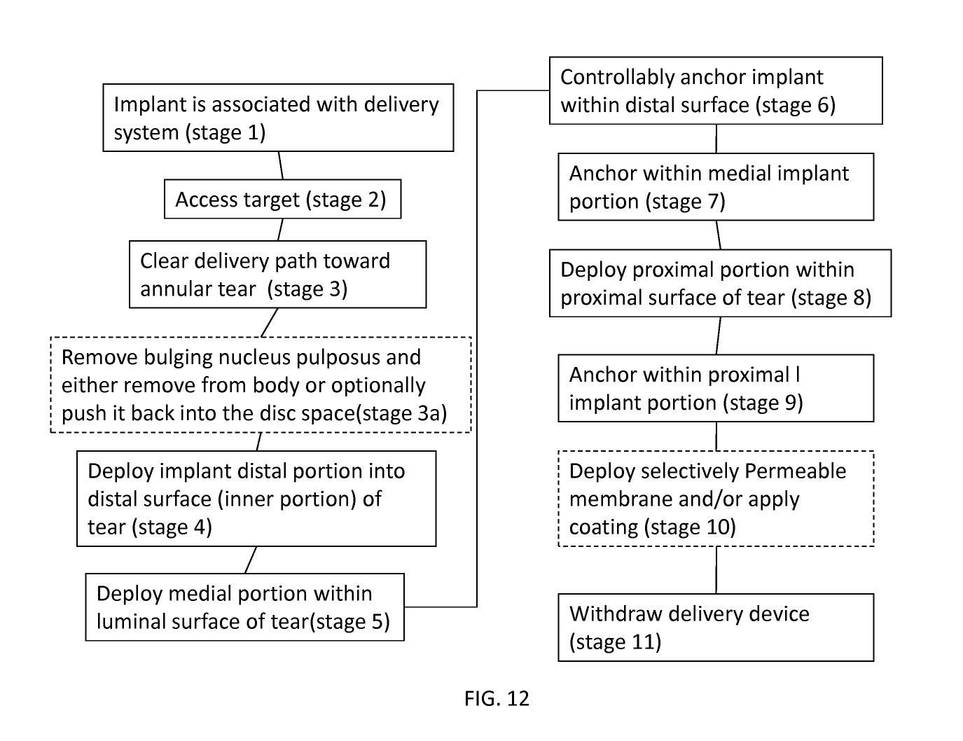

FIG. 12 shows a flowchart of an optional method for delivery an annular tear implant according to optional embodiments of the present invention as described in FIGS. 2-9 above and that may be further understood with reference to FIG. 1A-B.

First in stage 1 an implant for example 100, 110, 120, 130, 140, 150, 160, 170 provided in its folded, crimped and/or minimized formation within a delivery sheath (not shown) is associated with an implant delivery system for example including but not limited to a delivery catheter, guiding catheter, sheath tube, endoscopic working channel, endoscope, or the like. Optionally and preferably the implant is further associated with a guide wire and/or pusher or the like means for urging and maneuvering and controlling the implant into the implantation site optionally and preferably within the delivery system. Next in stage 2 access to implantation site 15 is gained while in stage 3 a delivery route is cleared toward annular tear implantation site 15 in preparation for implantation. Next in optional stage 3a removal of any bulging nucleus pulposus matter is performed, for example a partial discectomy, and is optionally vacated from the implantation site or placed within the confines of the annulus fibrosus; while the delivery site 15 is optionally shaped in preparation for implantation.
Next in stage 4 the distal portion of the delivery system, preferably comprising at least one implant, is positioned within the implantation site 15 preferably at about distal surface 24, preferably the optionally within the nucleus pulposus disc space 22. Once in position at distal surface 24 the implant is urged distally out of its sheath, optionally with a guiding wire and/or pusher and through the delivery system distal end and into implantation site 15 at distal surface 24. Preferably the distal portion of implant then unraveled from its crimped state, optionally and preferably exposing distal anchoring member, for example 102d, 112d, 122d, 132d, or 142d allowing them to expand.
Next in stage 5 implant body 104, 114, 124, 134, or 144 is exposed out of its delivery sheath and delivery system and placed within the luminal and/or medial surface 28 of implantation site 15. Optionally the delivery system and implant sheath are retracted proximally to expose implant body within medial surface 28.
Next in an optional and preferably stage 6 distal anchoring is provided in a controllable and reversible manner. Optionally and preferably distal anchoring members are anchored along the distal surface 24, most preferably without protruding into the lumen of nucleus pulposus 22. Optionally and preferably once exposed distal anchors are advanced distally and controllably folded back and/or curved proximally forming a hook like anchor that may be associated with distal surface 24. Most preferably distal anchors are positioned such that they conform to the shape of distal surface 24 gap formed by annular tear 15.
Next in an optional stage 7 an optional implant comprising anchors along its body contour may be anchored to the medial surface. Optionally, once in place within lumen 28 implant body may be radially expanded to fit and conform to the shape, contour size, of implantation site 15, for example with a balloon catheter and/or balloon over a guide wire or the like. Optionally radial expanding of the implant body may be done in a stepwise manner according to different portion of the implant body for example proximal and/or medial, and/or distal.
Next in stage 8 the proximal anchors are exposed form within the delivery system and implant sheath and controllably anchored to the proximal surface 26 of the implantation site 15. Next in an optional anchoring of proximal anchors is provided in optional stage 9. Optionally individual proximal anchors 102p, 112p, 122p, 132p, 142p, may be controllably and reversibly placed within proximal surface 26. Optionally and preferably once exposed proximal anchors are advanced distally and controllably folded back and/or curved distally forming a hook like anchor that may be associated with proximal surface 26. Most preferably proximal anchors are positioned such that they conform to the shape of distal surface 26 gap formed by annular tear 15.
Next in optional stage 10 an optional selectively permeable sealing membrane, for example 138m, 155, is delivered to proximal implantation surface 26 preferably through delivery system and associated with the implant's proximal portion 138, 158. Optionally a membrane is provided for with by coating the proximal delivery site 26 following proximal anchoring. Finally in stage 11 the delivery system is evacuated from the delivery site 15.

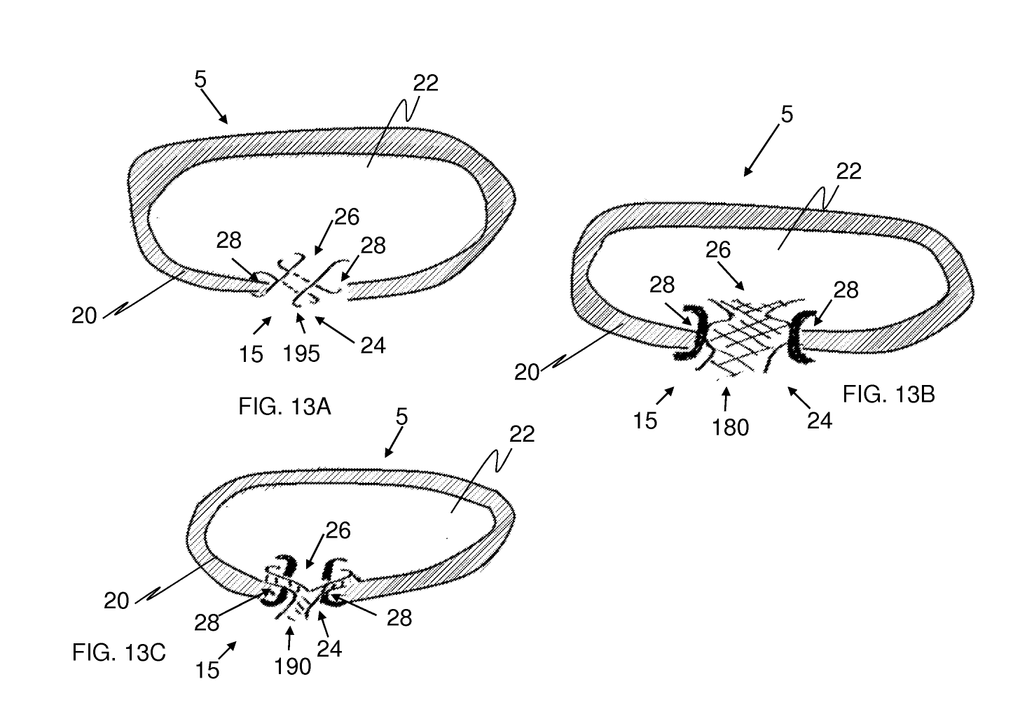

FIG. 13A-C provide a depiction of optional implants according to the device and method of the present invention following implantation within implantation zone 15 as described herein above. FIG. 13B depicts implant 180 of FIG. 10 within implantation zone 15. FIG. 13C depicts implant 190 of FIG. 11 implanted within implantation zone 15. FIG. 13A provides a depiction of an optional double lumen ‘X’ shaped implant 195 according to optional embodiments of the present invention.

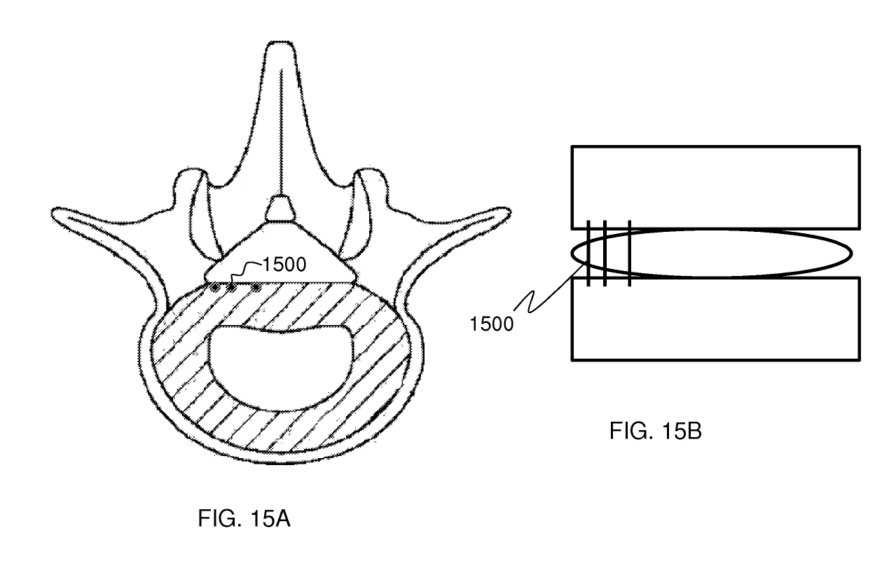

An optional non limiting embodiment of the present invention for a bar-like implant, for example 1500 as depicted in FIG. 15A-B, may optionally provide for coupling at least two or more spinal structures as shown in FIG. 15, with a at least one and more preferably a plurality of implants. Optionally, each bar extending from the inferior part of the endplate of the vertebra above the disc to the superior endplate of the vertebra below the disc, spanning the defect in the annulus fibrosus. Optionally individual bar-like implant 1500 may be anchored into the each endplate for example by a hook (not shown), nail, screw, or suture anchor, or a small metallic ball. Optionally, when juxtapositioned next to each other, individual bar-like implants 1500 constitute a barrier to solid tissue from being expelled from the disc space, while optionally and preferably allowing free passage of fluids. Optionally composition of each bar-like member 1500 member being of one of the materials previously having intrinsic flexibility, and allowing flexibility of the whole implant to move in all planes along with spinal column. Optionally at least one or more single or plurality of bar-like implants (1500) members in this embodiment may be optionally associated with and/or covered with a membrane preferably to increase its ability to prevent passage of material out of the disc space.

While the invention has been described with respect to a limited number of embodiments, it will be appreciated that many variations, modifications and other applications of the invention may be made.

1-37. (canceled)

38. An implant for repairing a tear in an annulus fibrosus of a spinal inter-vertebral disc wherein said implant comprises:
a body having a distal portion, a proximal portion and a medial portion, said proximal portion connected to said distal portion by said medial portion; and
at least one unidirectional sealing membrane,
wherein said at least one unidirectional sealing membrane is arranged to provide sealing for said tear,
wherein at least two of said distal portion, proximal portion and medial portion each comprise at least two anchors, and
wherein said tear comprises a distal surface, medial surface and a proximal surface.

39. The implant of claim 38, wherein said implant is flexibly and securely implanted within said tear along at least two surfaces chosen from the group consisting of said distal surface, said medial surface and said proximal surface.

40. The implant of claim 39, wherein said implant is anchored within said tear along said proximal surface and said distal surface.

41. The implant of claim 38, wherein said implant is anchored within said proximal surface, said medial surface and said distal surface.

42. The implant of claim 38, wherein said anchors are controllably maneuvered and disposed with said tear.

43. The implant of claim 38, wherein said anchors are controllably folded to form a hook like structure.

44. The implant of claim 43, wherein said anchors are disposed along at least one of said distal portion and said proximal portion.

45. The implant of claim 38, wherein said implant body is controllably shaped during implantation to securely and flexibly fit within said tear.

46. The implant of claim 45, wherein said implant body is adapted to comply with the anatomy and shape of said tear.

47. The implant of claim 38, wherein said implant body is provided in the form of at least one of: wire mesh, wire braid, screw, hollow screw, wire, bifurcation wire, extensions interconnected wire frame, spherical wire frame, dual sphere wire frame, stentlike mesh, stent-like tubular structure, elongated rods, spring and group of wires or rods with a flexible core.

48. The implant of claim 38, wherein said membrane is associated with at least one portion of said implant body chosen from the group consisting of said proximal portion, said distal portion, said medial portion and said anchors.

49. The implant of claim 48, wherein said membrane is disposed along an internal or external surface of said implant body.

50. The implant of claim 38, wherein said medial portion of said body comprises at least two members.

51. An implant for repairing a tear in an annulus fibrosus of a spinal disc, said implant comprising a body having a distal portion, a proximal portion and a medial portion,
said proximal portion connected to said distal portion by said medial portion,
wherein at least one of said distal portion, proximal portion and medial portion comprises at least two anchors,
wherein at least one of said distal portion, proximal portion and medial portion comprises at least one unidirectional sealing membrane, said at least one unidirectional sealing membrane arranged to provide sealing for said tear, and
wherein said tear comprises a distal surface, medial surface and a proximal surface.

52. The implant of claim 51, wherein said implant is provided from materials chosen from the group consisting of nitinol, stainless steel 316, memory shape polymers, beryllium copper alloys, cobalt-chromiummolybdenum alloy, cobalt chrome alloy and biological tissue.

53. The implant of claim 52, wherein said implant may be provided in a crimped and/or minimal profile prior to implantation and may be deployed to its full sized within the implantation site.

54. An implant for repairing a tear in an annulus fibrosus of a spinal disc, said implant comprising a body having a first and a second support member,
wherein at least a portion of said second support member lies within the lumen of said first support member,
wherein each support member comprises a distal portion, a proximal portion and a medial portion, said proximal portion connected to said distal portion by said medial portion,
wherein at least one of said distal portion, proximal portion and medial portion comprises at least two anchors,
wherein at least one of said distal portion, proximal portion and medial portion comprises at least one unidirectional sealing membrane, said at least one unidirectional sealing membrane arranged to provide sealing for said tear, and
wherein said tear comprises a distal surface, medial surface and a proximal surface.

55. The implant of claim 54, wherein said first support member has a substantially tubular shape along its medial portion and said second support member has a substantially Y shape.

56. The implant of claim 54, wherein said first and second support members associate with one another to form an X shape.

57. The implant of claim 54, wherein said first support member has a substantially tubular shape along its medial portion and said second support member has a substantially hourglass shape.
